(19) United States
(12) Patent Application Publication
(10) Pub. No.: US 2015/0216920 A1
(43) Pub. Date: Aug. 6, 2015

(54) INCENSE EXTRACT FRACTIONS AND USES THEREOF

(71) Applicant: ABOCA S.P.A. SOCIETA' AGRICOLA, Sansepolcro AR (IT)

(72) Inventor: Valentino Mercati, Sansepolcro (IT)

(21) Appl. No.: 14/429,471

(22) PCT Filed: Sep. 20, 2013
(86) PCT No.: PCT/IB2013/058690

§ 371 Date: Mar. 19, 2015

(30) Foreign Application Priority Data
Sep. 21, 2012 (IT) RM2012A000453

(51) Int. Cl. A61K 36/324

(52) U.S. Cl. CPC A61K 36/324; A61K 2236/333

(57) ABSTRACT

The present invention relates to a new incense extract wherein only traces of boswellic acids and resins are present, a process for the preparation of such extract, compositions comprising it, and the use of the extract and of the compositions for the protection of the skin or the mucous membranes.

The present invention relates to new incense extract fractions in which only traces of boswellic acids and resins are present, a process for the preparation of such fractions, compositions comprising them, and the use of the extract fractions and the compositions for the protection or the repair of the skin or the mucous membranes.

### STATE OF THE PRIOR ART

Incense is the name generically given to oleo-gum-resins secreted by various shrub plants growing in India, in the Southern regions of the Arabian peninsula and of the coasts of Eastern Africa opposite thereto; the most relevant resin, belonging to genus Boswellia, is Boswellia sacra.

Incense is therefore an oleo-gum-resin of plant origin, secreted by plants physiologically or, more often, in response to mechanical traumas (tappings, cuts) or to a stress (pathogen attack), comprised of a complex group of solid translucid substances, occasionally liquid, insoluble in water, soluble in alcohol, acetone, ether and chloroform, mainly comprised of boswellic acid and olibanoresene, of an essential oil (5%), a water-soluble gum (20-30%) and bassorine.
Said resin contains complex mixtures of aliphatic alcohols or acids, lignans, resinic acids, resinotannols, esters and resens (originating from essential oil terpenes polymerizing and oxidizing processes), etc.
Up to 16% of the resin is essential oil, alpha-thujene and p-cymene being the major constituents thereof. Finally, four are the pentacyclic triterpenic acids present in the essential oil, beta-boswellic acid being the main one.
Boswellic acids, present in incense, are the active principles giving thereto anti-arthritic, antiseptic, expectorant, astringent and stimulant properties, and making it indicated, e.g., in the treatment of asthma, irritable bowel syndrome, arthritis and Crohn's disease.

Commonly, the dry or lyophilized extract of incense obtained by extraction with hydroalcoholic mixtures contains 10 to 30% by weight, normally about 22% by weight of total boswellic acids, and the various known extraction methods are focused on improving the yield of these components, or on making such components more useful for therapeutic purposes.

### SUMMARY OF THE INVENTION

The present invention relates to incense extract fractions substantially depleted in the known pharmacologically active components represented by boswellic acids, of which only traces remain, and depleted also in resins, and to a process for obtaining such extract fractions. The extract fractions according to the invention may be used for a purpose completely different from that for which the incense extract is used in the state of the art, i.e. for the protection or the repair of the mucous membranes or the skin. Said use can be realized thanks to the protective activities of the remaining components, in which the extract fractions are enriched of the 15-25% with respect to the initial product thanks to the removal of oleo-resin and boswellic acids therefrom. The extract fractions of the invention therefore provide a new source of material of plant origin useful for the protection of the mucous membranes and of the skin, wherein the pharmacological effects attributable to the components known as active principles are substantially reduced or eliminated.

Said condition is particularly desirable when material of natural or plant origin are to be used substantially for actions of a mainly mechanical type, e.g. for cooperating with a pharmacological therapy, minimizing the presence of substances with pharmacological activities in the material of plant or natural origin in order to minimize possible undesired interactions with drugs, nevertheless exploiting the typical feature of substances of plant origin, of having a vast number of components besides the pharmacologically active ones commonly used.

The use of extracts of plant or natural complex substances depleted in pharmacologically active principles entails the dual advantage of providing a material with a mainly mechanical protective action (mucous membrane protection or skin protection) and of enabling a concrete and useful use of materials normally discarded in extraction processes of plant or natural active principles.

The present invention therefore relates to incense extract fractions depleted in total boswellic acids, wherein said boswellic acids have a concentration in weight percentage 1.3 or 0.1%, their use in the protection and/or in the repair of the skin or the mucous membranes, compositions comprising such extract and their use in the protection and/or in the repair of the skin or the mucous membranes and a process for the preparation of such extract fractions and of such compositions. The invention also relates to a process for the preparation of incense extract fractions as defined above, comprising the following steps:

a. grains of incense, optionally fragmented, are subjected to three or more steps of extraction in ethanol at decreasing concentrations, comprising at least one extraction with 85% ethanol, one extraction with 13% ethanol and one extraction with 3% ethanol;

b. the alcoholic extracts obtained with 13% ethanol and 3% ethanol in step a. are gathered and subjected to decantation and/or centrifugation;

c. the supernatant obtained after step b) is concentrated, obtaining a concentrated aqueous extract fraction, defined herein as fraction C, wherein said fraction C comprises boswellic acids at a concentration expressed as weight percentage 1.3%

d. optionally, said fraction C is filtered;

f. the permeate obtained in step d. is collected as fraction D and comprises boswellic acids at a concentration expressed as weight percentage 0.1%.

### SHORT GLOSSARY

NMWCO=Nominal Molecular weight Cut-off

PERMEATE: extract that has passed through the semipermeable filtration or ultrafiltration membrane

RETENTATE: extract that did not pass through the filtration or ultrafiltration membrane

FILTRATION: Filtration technique on semipermeable membrane characterized by pores having sizes of 30 to 0.1 micrometers

ULTRAFILTRATION: Filtration technique on semipermeable membrane, characterized by pores having sizes of 100,000 daltons (0.1 um) to 1,000 daltons (about 0.005 um)

Weight percentages according to the present description are reported with respect to the weight of the extract or of the fractions of interest measured after lyophilization.

### DETAILED DESCRIPTION OF THE FIGURES

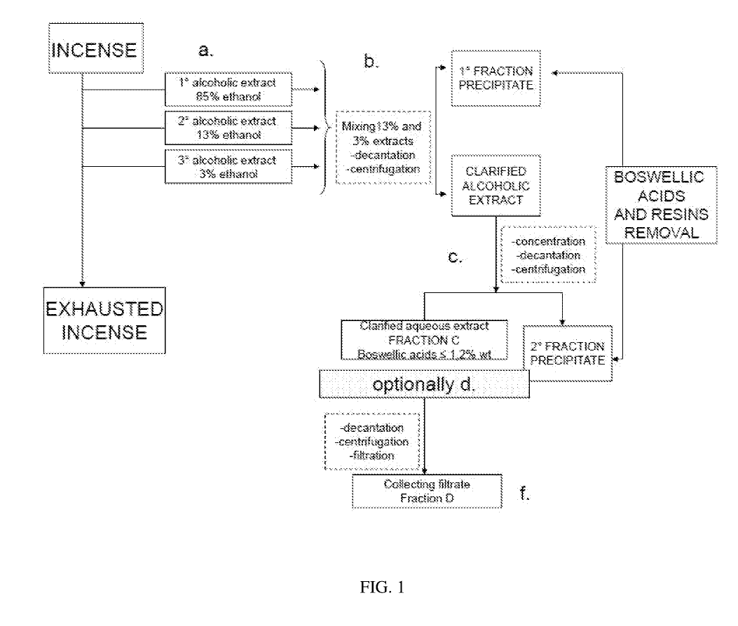

FIG. 1 shows a block diagram of an embodiment of the process of the invention in which ultrafiltration step e is not present.

FIG. 2 shows a block diagram of step e.

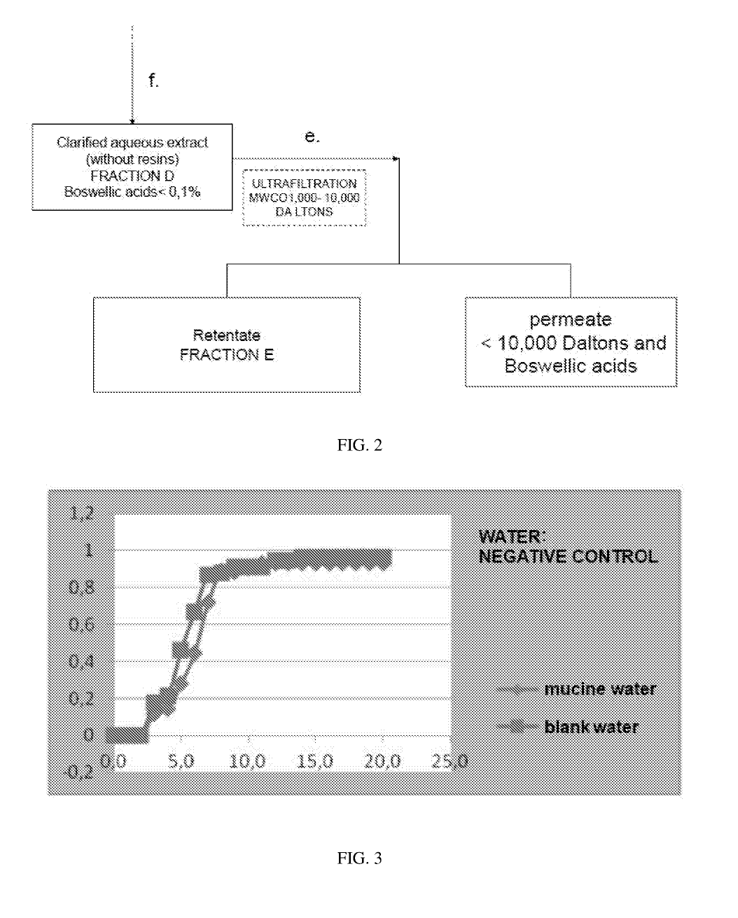

FIG. 3 shows a graph of mucoadhesion on an inclined plane (test described below) in which the tested substance is water.

Rhombs denote results obtained on an inclined plane with mucin and water, whereas squares denote those obtained on the control (blank) in which water is tested on an inclined plane without mucin.
The figure shows that for water, a non-mucoadhesive substance, the measurements of the sliding on the inclined plane with and without mucin are practically overlappable.

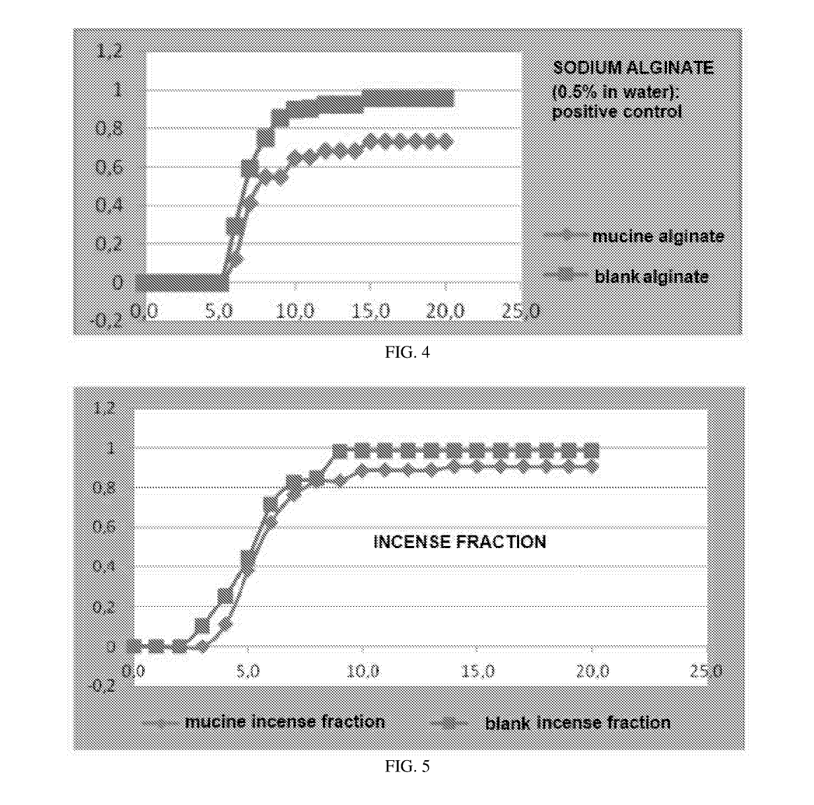

FIG. 4 shows a graph of mucoadhesion on an inclined plane (test described below) in which the tested substance is sodium alginate, a substance with known mucoadhesive properties.

Rhombs denote results obtained on an inclined plane with mucin and sodium alginate, whereas squares denote those obtained on the control (blank) in which sodium alginate is tested on an inclined plane without mucin.
In the case of the sample consisting of sodium alginate, a remarkable difference in behavior is witnessed in the presence and in the absence of mucin.

FIG. 5 shows a graph of mucoadhesion on an inclined plane (test described below) in which the tested substance is the incense extract fraction C according to the present description in order to verify its mucoadhesive properties. Rhombs denote results obtained on an inclined plane with mucin and incense extract according to the present description, whereas squares denote those obtained on the control (blank) in which the incense extract fraction C according to the present description is tested on an inclined plane without mucin.

It may be noted how the extract fraction has a behavior similar to sodium alginate; a remarkable difference in behavior is witnessed in the presence and in the absence of mucin, thereby resulting with mucoadhesive features.

### DETAILED DESCRIPTION OF THE INVENTION

As indicated above, in the present description incense extract fractions depleted in total boswellic acids are provided, wherein said boswellic acids have a concentration in weight percentage 1.3%, or even 0.1%, such as of about 1.2% or 0.05%.

By applying the extraction process object of the invention, described below, and separation techniques on semipermeable membrane, it was possible to obtain various incense extract fractions, some of which are depleted in resins and in boswellic acids and concomitantly surprisingly enriched in the remaining substances; said fractions showed an effective protective action on the skin and on the mucous membranes, as evident from the mucoadhesivity assay and barrier assay reported in the experimental section below.

In particular, the fractions isolated in the present invention exhibit barrier effect and mucoadhesion effect features greater than those of the initial incense extract from which they are separated.

With respect to the dry extracts of incense and to the incense as such, which comprises a minimum amount of boswellic acids between about 10 and 30% in weight, and with respect to the resinous extracts of incense, which comprise an amount of boswellic acids between 15 and 60%, the fractions of the present invention have merely traces of such active principles, as comprising an amount of boswellic acids 1.3% or even about 0.1% to about 0.05% in weight.

Evidently, such percentages are in practice traces of boswellic acids that cannot be accountable for the mucoadhesive and barrier properties exhibited by the fractions of the invention.

Since the extract fractions are parallelly depleted also in resins, the latter being them also almost completely eliminated from the former in the same steps of elimination of boswellic acids, the mucoadhesive effect and the barrier effect observed are not ascribable to resins either.

The extract fractions of the invention are therefore useful for their mucoadhesion and barrier effect properties in all those cases in which the protection of skin or mucous membranes is needed or desirable; those may be cases in which a drug that attacks mucous membranes or skin has to be administered, therefore in order to prevent the damaging action of the drug, or in those cases in which the protection of a compromised or even partially compromised skin or mucous membrane is preferable or desirable, so as to enable a better and quicker healing thereof, defending it from further aggressions, or in those cases in which an individual has a chronic disorder in which skin or mucous membranes sustain irritations or alterations, therefore a barrier effect can prevent or limit damages on the skin or the mucous membrane, and a mucoadhesive effect can enable the barrier to persist longer over the zone to be protected. In particular, the fractions can also be used as protective and mucoadhesive carriers for active pharmaceutical principles other than boswellic acids.

In outlining the protection effect of mucous membranes, in the present description it is meant that such mucous membranes can be the oral mucosa, gastric mucosa, intestinal mucosa, the nasal mucosa, the vaginal mucosa, the uterine mucosa, the rectal mucosa.

The present invention also provides a process for the preparation of extract fractions as described above, said process comprising the following steps:

a. grains of incense, optionally fragmented, are subjected to three or more steps of extraction in ethanol at decreasing concentrations, comprising at least one extraction with 85% ethanol, one extraction with 13% ethanol and one extraction with 3% ethanol;

b. the alcoholic extracts obtained with 13% ethanol and 3% ethanol in step a. are gathered and subjected to decantation and/or centrifugation;

c. the supernatant obtained after step b) is concentrated, obtaining a concentrated aqueous extract fraction, defined herein as fraction C, wherein said fraction C comprises boswellic acids at a concentration expressed as weight percentage 1.3%;

d. optionally, said fraction C is filtered; and

f. the permeate obtained in step d. is collected as fraction D and comprises boswellic acids at a concentration in weight percentage 0.1%.

According to the invention, the above-described process can be carried out by performing in step a. at least three, or at least four steps in decreasing ethanol concentrations, in which the decreasing ethanol concentrations go from about 85% ethanol to about 3% ethanol.

For instance, step a. of the process may be carried out by performing a step with about 85% ethanol, and the residue obtained from this step is then subjected to a step with about 13% ethanol, then performing a further step of extraction of the residue obtained with the extraction in 13% ethanol, in about 3% ethanol.

Or, step a. of the process could be carried out by performing, prior to the step with about 85% ethanol, also steps at higher ethanol concentrations, like, e.g., by performing also an extraction with about 96% ethanol.

Each of the alcoholic extracts obtained from the steps in 13% and in 3% will then be used in step b. of the process.

The alcoholic extracts obtained from the steps in 13% and in 3% in step a. are gathered to form a new alcoholic extract and subjected to decantation and/or centrifugation in step b. of the process.

As already indicated above, the new alcoholic extract obtained at step b. could be decanted and/or centrifuged so as to form a precipitate and a supernatant.

Decantation could be performed, e.g., 1 to 72 hours, and could be followed or replaced by one or more centrifugations to about 3000-4000 (e.g., about 3500) rpm for a period comprised between 1 and 10 minutes, e.g. about 5 minutes.

The supernatant obtained after this step/s is then collected and concentrated by alcohol evaporation, subjecting it, in step c., to alcohol evaporation by standard techniques, like, e.g., by use of a thin film distillation system, or a batch concentration system according to standard protocols commonly used by the technician in the field.

The result of this evaporation is a concentrated aqueous solution, also referred to as fraction C, that has a concentration of boswellic acids expressed as weight percentage 1.3% i.e. of about 1.2%, and is characterized by a barrier effect, measured according to the methods described below, of about 75%, compared to the 23% of the control standard hydroalcoholic extract (extract obtained by 60% hydroalcoholic extraction and subsequent lyophilization) and by a mucoadhesion, measured with inclined-plane adhesion assays, equal to >50% compared to the near-absence of the control extract mucoadhesion, which in the assays appears to be nil.

Fraction C may then be, in step d. of the process, optionally subjected to decantation before being filtered.

The concentrated aqueous solution, or fraction C, may be subjected to decantation for a period comprised between 1 and 72 hours, and the supernatant is then recovered and subsequently filtered, thereby obtaining a clarified solution.

Alternatively, the concentrated aqueous solution, or fraction C, is subjected to one or more filtration steps (using filters with a decreasing cutoff to about 30, 0.5 and 0.1 micrometers) thereby obtaining a clarified solution, also referred to as fraction D. The filtration could be performed, e.g., on panel filters with a cutoff from about 30 to a cutoff of about 0.1 micrometers, and the permeate obtained is collected in fraction D.

The filtrate obtained as described above (fraction D) is recovered in step f. of the process and constitutes an incense extract fraction comprising boswellic acids at a concentration expressed as weight percentage not higher than 0.1% i.e. 0.1% in weight.

In particular, fraction D is characterized by a concentration of boswellic acids expressed as weight percentage of about 0.05%.

All of the above-described fractions are then normally subjected to a lyophilization or drying step.

Optionally, the process of the invention could comprise a further treatment of the filtrate obtained at the above-described step d. (fraction D) before collection of the desired extract.

In this further embodiment, the process will comprise a step e. wherein the filtrate or the supernatant obtained in d. is subjected to one or more steps of ultrafiltration on membrane with a cutoff of from 1,000 to 10,000 Daltons, e.g. of about 10,000 daltons, and the retentate is collected as fraction E.

Of course, the fractions of the invention will contain only traces also of other substances of terpenoid structure present in the starting product, besides of boswellic acids.

In this further step, the aqueous extract fraction (or fraction D, result of the filtration) is subjected to one or more steps of ultrafiltration on membranes with a molecular weight cutoff (NMCWO) of from 1,000 to 10,000 daltons, e.g. of 10,000 daltons. The retentate fraction thus obtained is substantially free from boswellic acids and resins and substances of terpenoid structure, whereas it is enriched in substances having a protective action and can be used as is or subjected to a lyophilization process to provide a lyophilized extract fraction useful for formulations having therapeutic employ for internal and/or external use.

The above-described fractions have the features reported in Table 1, where they are compared with a control extract (obtained by 60% hydroalcoholic extraction of incense grains and subsequently lyophilized), i.e. a type of extract commonly referred to as “incense extract” in the state of the art.

TABLE 1

| | | BA, % of | |
|---|---|---|---|
| | | inhibition | MUCOADHESION |
| | | IL-6 | ASSAY ON |
| | BOSWELLIC | RELEASE | INCLINED PLANE |
| | ACIDS HPLC | (conc. 20 | % (conc. 10 |
| Sample | % | mg/ml) | mg/ml) |
| REFERENCE | 22.39 | 23 | 0 |
| INCENSE | | | |
| EXTRACT | | | |
| (60% ethanol) | | | |
| Incense Fraction C | 1.20 | 75 | >50 |
| LYOPHILIZED | | | |
| Incense 3rd | 0.05 | 57 | >50 |
| Fraction D | | | |
| LYOPHILIZED - | | | |

The process according to the present invention, wherein the order of the steps will be a, b, c, and optionally d, f, enables therefore to obtain incense extract fractions as described above, depleted in oleoresin and therefore in boswellic acids and surprisingly rich in substances having a protective action. The above-described process also eliminates resins via the reagents and steps used, as evident to the technician in the field.

Therefore, the extract fractions of the invention will also be substantially free from resins that will be found in the precipitate of step d and in the retentate of the ultrafiltration step.

The present invention also relates to a composition comprising one or both the extract fractions in any one of the above-described embodiments, useful in the protection and/or in the repair of the skin or the mucous membranes.

Said protection, of course with reference both to the extract per se and to the composition, could be a preventive protection or a protection of compromised or partially compromised skin or mucous membranes.

For instance, the composition or the extract can, by virtue of their barrier effect, facilitate the repair of damaged skin or mucous tissue, with entailed restoration of a healthy and elastic skin or mucous membrane.

The skin lesions according to the present invention are, e.g., lesions that may involve also tissue underlying the skin and in which no open wounds are present, or, when the fractions of the invention are associated to cicatrizing, anti-inflammatory, antibiotic active principles, and other active principles commonly used in the treatment of open wounds, the fractions or the compositions of the invention can be used also to cooperate with the treatment of lesions having open wounds.

By skin lesions not involving the presence of open wounds, according to the present description, are meant those lesions in which the superficial layer of the skin, and the underlying layers, though not being wounded, are particularly fragile, irritated and damaged.

Non-limiting examples of this type of lesions are represented by first-degree burns, first-degree decubitus lesions, pressure lesions, newly-cicatrized rashes, wounds or burns, irritations, erhytemas.

The composition or the extract fractions of the invention (C and/or D) could then be used for the treatment or the prevention of skin lesions not involving the presence of open wounds, or in the prevention or slowing down of worsenings of the same or, in association with appropriate active principles (not comprising boswellic acids), for the treatment of lesions involving the presence of open wounds. The compositions or the fractions according to the invention could advantageously comprise further components, e.g. of plant origin, having, e.g., emollient, digestive, prokinetics, cholagogue, carminative, prebiotic, relaxing, excipient, preservative, humectant properties, not comprising boswellic acids.

When the composition or the extract fractions of the invention are used for the protection of the skin, the application thereof will be topical. The embodiments of the compositions for topical use are reported hereinafter in the description.

The composition according to the invention could be made, e.g., as oil-in-water emulsion, water-in-oil, oil-in-gel or gel-in-oil emulsion, multiple emulsions, sprays, and anhydrous formulations (salve, gel, paste, cream, ointment) and will comprise one or more excipients suitable for the making of the desired end form.

Such excipients could be, e.g., emulsifying agents (cetearyl alcohol, cetearyl glucoside, hydrogenated castor oil), rheological additives, antioxidants (like, e.g., vitamins, tocopherols or other antioxidants known in the field).

The emulsifying agent could be a surfactant, which by lowering the interfacial tension decreases the free energy of the system; alternatively, also non-surfactant substances can be used, such as gum arabic, gelatin, hydrophilic colloids or finely subdivided powders (e.g., talc). In one embodiment, the excipients could be present at an overall concentration in weight comprised between 3 and 8%, such concentration being of course indicative, taking into account that anyhow the technician in the field will know how to adapt the concentration of the necessary excipients according to the embodiment he/she intends to prepare without addition of inventive activity.

Moreover, the composition could comprise perfuming and/or coloring agents making its odor pleasant, like, e.g., one or more essential oils like, e.g., lavender essential oil, melaleuca, lemon, mint, orange essential oil, and/or coloring agents enabling, e.g., to easily recognize the zones of application of the composition, the coloring being, e.g., temporary, so as not to interfere with other, subsequent applications of the composition.

In one embodiment, said coloring and/or perfuming agents are comprised at an overall concentration in weight between 0.001 and 3%.

By “overall concentration in weight” it is meant the concentration in weight in the composition of the sum of the various excipients, or the concentration in weight in the composition of the sum of the various perfuming and/or coloring agents present in the composition.

As to the use of the composition in the protection of the skin, the invention also relates to medical devices like, e.g., medicated plasters, medicated gauzes, medicated bandages, medicated tissues, medicated tampons, medicated diapers (pads), i.e. plasters, gauzes, bandages, tissues, tampons or diapers (pads) which comprise, or be at least partly covered by, or be at least partly soaked with the described composition of the invention, or with the described fractions in the most appropriate form.

The gauzes could be made as fatty gauzes, and so the bandages and the plasters, by using the composition made in the form, e.g., of ointment, paste or cream. The tissues could be soaked with the composition in the form of emulsion of oil with water or gel, and the diapers (pads) or the tampons could be made by inserting the composition or the fractions in the suitable layers known in the field for the insertion of protective or anti-irritating compositions.

Such products, like, e.g., diapers, tampons (commonly called “sanitary napkins”) for women and pads for adult incontinence, are commonly used, e.g. in the babyhood/childhood-related field, in the feminine field and in the geriatric field, and the technician in the field will know where to insert the composition or the fractions described herein, and which embodiment be the most suitable one, with no need of specific teachings and by basing him/herself solely on techniques conventional in the field.

Such devices will be applicable over the part to be treated and/or protected by way of prevention.

As already indicated above, the composition of the invention may be a composition for topical use which could be realized in the form of oil-in-water emulsion, water-in-oil emulsion, multiple emulsions, sprays, and anhydrous formulations (salve, ointment, gel, paste, cream, spray,) according to techniques commonly used in the field. The formulation indicated herein as “spray” could be an anhydrous formulation or even an emulsion formulation, the form with an atomizer enabling also self-applications in zones harder to reach (like, e.g., the back), useful in all those cases in which the formulation is used, e.g., to alleviate sun rashes, erythemas, or skin irritations of various kind.

According to another embodiment, the fractions C and/or D or the composition of the invention could be used, thanks to their mucoadhesive and barrier effects, for the protection and/or for the repair of the mucous membranes.

In this case as well, such protection could be a preventive protection, e.g. in all those cases envisaging an administration of drugs having the side effect of attacking the mucous membranes, or in those patients exhibiting conditions of recurring irritation of the same. In other cases, the protection could be instead a protection for curative purposes or in order to avoid the worsening of irritated or partially compromised mucosal membranes. Finally, the composition or the fractions, thanks to their barrier and mucoadhesive effects, could actively facilitate and enable the repair of compromised or partially compromised mucosal tissues, by mechanically preventing the action of irritating or aggressive agents.

In these cases, the administration of the fractions or of the composition could be topical for all those mucous membranes on which a topical administration is possible (e.g. oral, rectal, vaginal, nasal mucosa), or oral in the cases in which said membranes be, e.g., intestinal or gastric mucosal membranes.

The compositions for the protection of the mucous membranes could therefore be made in the form of capsule, tablet, lozenge, granule, powder, syrup, elixir, hard gelatine, soft gelatine, suspension, emulsion, solution, suppository, cream, gel, spray, ointment, salve, paste, oil-in-water emulsion, water-in-oil emulsion, oil-in-gel emulsion, gel-in-oil emulsion.

In case of formulations for topical use, the above indications related to the compositions for the protection of the skin could be followed, or syrups, rinses, sprays could be made, e.g., for the protection of the mucous membranes of the mouth, throat, nose.

For application on the rectal mucosa, suppositories or enemas or microenemas could be used, with the excipients known to the technician in the field for the making of such compositions.

As already mentioned, the composition of the invention could be in the form of capsule, tablet, lozenge, hard gelatine, soft gelatine, granule, powder, syrup, elixir, suspension, emulsion. For oral administration the composition could be made in the form of daily unit dosages or of fractions of daily unit dosages (e.g. 2, 3, 4, 5, 6, or more capsules, tablets, lozenges, granule or powder single-doses, or gelatins could be taken over the day, according to the judgment of the doctor in charge), and may contain conventional excipients, including, e.g., binding agents, like gum arabic, gelatine, sorbitol, gum tragacanth, and/or polyvinylpyrrolidone; fillers, like lactose, sugar, corn starch, rice starch, calcium phosphate, sorbitol and/or glycine; tableting lubricants, like magnesium stearate, talc, polyethylenglycol and/or silica; disintegrants, e.g. potato starch; and moisturizers like sodium laurylsulphate. The tablets can be coated according to methods well-known in the standard pharmaceutical practice.

The composition could also be made in a liquid or semiliquid form, as a suspension, emulsion, solution for oral administration, and could optionally contain natural aromatizing agents giving a palatable taste thereto.

The composition in the form of powder or granule could be pre-metered in suitable containers and ready for use, either by ingestion as such or to be resuspended in an appropriate liquid such as water, tea, etc. In this case as well, the composition could contain natural aromatizing agents giving a palatable taste thereto.

Evidently, all of the above-indicated excipients could be used in a pharmaceutically acceptable grade.

In one embodiment, the composition as described herein, in any one of the above-indicated embodiments, could be in the form of pharmaceutical composition, i.e., comprise pharmaceutical-grade ingredients, or it could be or be introduced into a special-purpose food or into a medical device.

The composition according to the present description could be made in the form of pharmaceutical composition or of medical device according to any one of the classes described in Directive 93/42/EEC on medical devices (comprising also substances and not only “devices” in the mechanical sense of the term), or in the form of medical food, or in any form according to the regulatory provisions of the Country in which said composition will be produced.

The medical device or the medical food could also contain as ingredients other components comprising, e.g., combinations of vitamins, mineral salts and other substances aimed at diet supplementing.

The fractions or the composition of the invention are therefore useful for their mucoadhesion and/or barrier effect properties in all those cases in which the protection of skin or mucous membranes is needed or desirable, which may be cases wherein a drug which attacks the mucous membranes or the skin has to be administered, therefore in order to prevent or limit the damaging action of the drug, or in those cases in which the protection of a partially compromised skin or mucous membrane is preferable or desirable so as to enable a better and quicker healing thereof, defending it from further aggressions, or in those cases in which an individual has a chronic disorder in which skin or mucous membranes undergo irritations or alterations whereby a barrier effect can prevent or limit damages on the skin or the mucous membrane.

In a further embodiment, the fractions of the invention, alone or mixed, could be used as mucoadhesive and mucoprotective carriers in pharmaceutical formulations.

The invention also relates to a process for the treatment or for the prevention of the onset or the worsening of skin lesions involving or not involving the presence of open wounds, wherein such process comprises one or more applications of the composition of the invention or of the medical device comprising it once or more per day on the concerned part.

The application of the composition, for instance, could be repeated whenever needed (e.g. at each change of pad in case of prevention of incontinence-related rashes) or once, twice, thrice, four or more times per day in general.

The invention also relates to a method for the preparation of a composition according to any one of the embodiments described above, wherein incense extract fractions depleted in total boswellic acids, wherein said boswellic acids have a concentration in weight percentage 1.3% or even 0.1%, are mixed with at least one of an excipient and/or a substance of natural and/or plant origin having emollient, digestive, prokinetics, cholagogue, carminative, prebiotic, relaxing, excipient, preservative, humectant properties as described above, wherein these substances are not boswellic acids.

Among these, e.g., could be used one or more of: extract of gentian root, boldo leaves extract, milk thistle fruits extract, artichoke leaves extract, dandelion root extract, anise fruit extract, rosemary leaf extract, mint leaves extract, marjoram leaves extract, cumin fruit extract, coriander fruit extract, ginger root extract, fennel fruit extract, caraway fruit extract, charcoal, inulin.

Object of the present invention is also a process for the protective (preventive or curative) treatment of the skin or the mucous membranes, providing the administration of the fractions or the composition of the present invention to a patient in need thereof. Such administration could also be concomitantly with the administration of other drugs.

The absence of pharmacologically active principles of incense in the extract fractions or in the composition makes such products particularly suitable to administration concomitantly with other drugs, as a side effect of interaction between the fractions, or between the composition of the invention and the drug co-administered or concomitantly administered is markedly unlikely.

A non-limiting example of the process of treatment and/or of prevention of the skin or the mucous membranes could comprise the administration of a daily dosage, subdivided into a single dose or plural doses, described of the mixture or composition according to the present description, for a period of time comprised between one and six weeks, e.g. comprised between three and six weeks, or even for a period of time higher than six weeks, according to the judgment of the doctor in charge.

Such administration could precede the administration of the drug even for a prolonged period, so as to optimize the health state of the skin or of the mucous membrane to be treated.

The doctor in charge will know how to establish both the most appropriate dosage and the administration times also on the basis of the patient's health state, weight, gender and age.
One of the substantial differences between the structure of the skin and that of the mucous membranes is represented by the absence, in the mucous membranes, of a selective barrier such as the corneous layer. Oral mucous membranes contact with noxious or irritating substances present in the environment (pollutants, pathogenic microorganisms, etc.) can therefore cause a high penetration of said substances both inside the mucous membranes and in the related airways (bronchi, lungs, etc.) causing inflammatory and/or allergic pathologies.

The protective effect of the extract fractions of the present invention was assessed by mucoadhesion assays and barrier effect assays.

The former aim at evaluating the mucoadhesivity of the product under examination in order to establish whether such product has the ability to adhere to the mucous membranes and consequently exert a protective action.

For instance, two simple designs are available for evaluating mucoadhesivity of the product of interest.

Evaluation of Adhesion Percentage by Inclined Plane with Mucin.

Measurements are performed by an equipment comprised of a 45°-inclined plane, thermostated to 37° C.; below said plane a microbalance is set, which performs measurements at regular 1-s intervals and prints recorded measurements on paper. Performed measurements are then transcribed and reprocessed on Excel spreadsheet.

The inclined plane acts as support for the biological substrate, consisting of a mucin film set up by depositing, on a plexiglas plane kept horizontal, a volume equal to 3 ml of a suspension of porcine gastric mucin at an 8% concentration (w/w) in a phosphate buffer, pH 6.4.

The dispersion is then brought to dryness at the temperature of 44° C., so as to obtain a film of known surface area equal to 33.6 cm².

An exactly weighed amount of the extract according to the invention is dissolved in a solvent. The extract is then deposited on the measurement plane with a multichannel pipette at constant time and rate during 6 seconds.

The multichannel pipette is calibrated so as to meter the solution (or dispersion) over the work plane. The total amount is measured at each assay (the weight of said volume is measured before the assay and depends on the density of the sample under analysis).

The sample, which will fall on the microbalance at the end of the run, is loaded in the top part of the inclined plane, containing the mucin film, and let slide thereon. The amount of fallen sample is measured by recording the weight variation as a function of time. The assay as made herein lasts 40 seconds, though usually after 20 seconds there is no weighing variation.

Measurements in the absence of the mucin film on the inclined plane are also performed (measurements indicated as blank), with the same amounts of sample and under the same testing conditions.

Thus, it is possible to assess the intrinsic sliding properties of the sample, regardless of its ability to interact with the biological substrate.

The adhered % is calculated: defined as the amount of sample remained adhered to the mucin film or to the inclined plane (without mucin, blank) at the end of the measurement, normalized with respect to the amount deposited on the inclined plane.

Moreover, it is determined the percentage difference between sample amount adhered to the plane with mucin and sample amount adhered to the plane without mucin. The calculation is performed in accordance with the following equation:

% difference=(adhered mucin %−adhered blank %)/adhered % of the blank

where:
adhered mucin %=sample percentage remained adhered to the mucin film at the end of the measurement;
adhered blank %=sample percentage at the inclined plane at the end of the blank measurements.

Barrier Effect Assay

The barrier effect assay is a biological-type in vitro assay employed for assessing the ability of finished products and/or raw materials to protect, through the formation of a thin “insulating” layer, mucous membranes and skin from contact with environmental contaminants (powder, pollens, microorganisms, etc.).

The assay was developed to simulate in vitro the action exerted by products that are applied on skin and/or mucous membranes with the aim of creating a protective film against external aggressors.

The design exploits the principle whereby cells subjected to contact with an inflammatory agent produce and secrete pro-inflammatory mediators (cytokines) in the extracellular environment in an amount correlated to the degree of inflammation caused; within a certain range, a direct proportionality exists between concentration and times of exposure to the inflammatory agent and amounts of released cytokines.

The experimental design adopted provides two chambers physically separated by a semipermeable membrane (0.4 μm pores). Cells are seeded in the bottom chamber, whereas the top chamber accommodates the inflammatory agent; on the semipermeable membrane separating the two chambers a thin film of the sample under analysis is stratified to highlight, if present, a barrier effect to the free transit of the inflammatory agent.

The semipermeable membrane allows transit of the inflammatory agent in the bottom chamber and constitutes the support on which the sample to be tested is stratified. Depending on the “insulating” ability of the sample, a decrease of LPS migration from the top chamber to the bottom one will be had (therefore a lesser stimulation of cells to cytokine production).

The examples reported below aim to indicate some embodiments of the invention or to provide details on technical procedures, without however being limitative thereof. Therefore, what indicated in the examples is to be considered as part of the possible embodiments of the invention.

### EXAMPLES

### Example 1

### Preparation of Extract Fractions

Incense grains were fragmented and subjected to three steps of extraction in ethanol at decreasing concentrations; performing on the incense a step with 85% ethanol, a step with 13% ethanol, a step with 3% ethanol.

The alcoholic extracts obtained with the 13% and 3% steps as described were gathered and subjected to centrifugation for a time of 1-5 minutes at a rotation rate equal to 3500 rpm; the supernatant was recovered, and concentrated by ethanol evaporation with use of a thin film distillation system according to the standard protocol, providing the feeding of the extract to be concentrated at a rate of about 500 l/h; Operation was by setting a residual vacuum of 0.6-0.8 bars and the fluid heating the evaporation walls was set at 140° C., thereby obtaining, after ethanol elimination, a concentrated aqueous extract fraction corresponding to fraction C, comprising boswellic acids at a concentration, expressed as weight percentage, equal to 1.2%.

The fraction thus obtained was also filtered on panel filters with a cutoff decreasing from 30 to 0.1 micrometers (30, 0.5 and 0.1).

The filtrate thus obtained (fraction D), comprised boswellic acids at a concentration expressed as weight percentage of 0.5%.

### Example 2

### Assessment of Mucoadhesive Ability of Extract Fractions of the Invention on an Inclined Plane

Measurements were performed by means of an equipment consisting of a 45°-inclined plane, thermostated to 37° C.; below said plane, a microbalance was set which performed measurements at regular 1-s intervals and printed recorded measurements on paper. Performed measurements were transcribed and reprocessed on Excel spreadsheet.

The inclined plane acts as support for the biological substrate, consisting of a mucin film. The mucin film was set up by depositing, on the plane consisting of plexiglas kept horizontal, a volume equal to 3 ml of a suspension of porcine gastric mucin at an 8% concentration (w/w) in a phosphate buffer, pH 6.4.

The dispersion was brought to dryness at the temperature of 44° C., so as to obtain a film of known surface area equal to 33.6 cm². Amount, concentration and deposition plane were defined during process development.

An exactly weighed amount of the extract fractions of the invention of interest and of the control extract was dissolved in solvent (0.5% water). The extract was deposited on the measurement plane with a multichannel pipette at constant time and rate during 6 seconds.

The multichannel pipette was adjusted so as to meter 280 microliters of solution (or dispersion) over the work plane. The total amount measured at each assay is of 1.12 ml per sample (the weight of said volume is measured before the assay and depends on the density of the sample under analysis).

The sample, which would fall on the microbalance, was loaded in the top part of the inclined plane, containing the mucin film, and let slide thereon.

The amount of fallen sample was measured by recording the weight variation as a function of time.

The assay lasted 40 seconds, however it is stressed that after 20 seconds there was no weighing variation.

Measurements in the absence of the mucin film were also performed (measurements indicated as blank) with the same amounts of sample and under the same testing conditions, but without the mucin layer deposited on the plane. Thus, it was possible to assess the intrinsic sliding properties of the sample, regardless of its ability to interact with the biological substrate.

The adhered % was calculated: defined as the amount of sample remained adhered to the mucin film or to the inclined plane (without mucin, blank) at the end of the measurement, normalized with respect to the amount deposited on the inclined plane.

Moreover, it was determined the percentage difference between sample amount adhered to the plane with mucin and sample amount adhered to the plane without mucin. The calculation was performed in accordance with the following equation:

% difference=(adhered mucin %−adhered blank %)/adhered % of the blank.

### Where:

adhered mucin %=sample percentage remained adhered to the mucin film at the end of the measurement
adhered blank %=sample percentage at the inclined plane at the end of the blank measurements.

### Analyzed Samples:

The measurements were performed for the following samples:
Water: used as negative control, i.e. as a solution without mucoadhesion
0.5% sodium alginate in water used as positive control, i.e. as a substance with known mucoadhesive properties
Incense sample:
   Reference lyophilized extract (in 60% ethanol)

lyophilized fraction C: 0.1% solution in water

lyophilized fraction D: 0.1% solution in water

Obtained data show that fractions C and D selected in the present invention acquire mucoadhesive properties absent in the control extract (identical amounts of each lyophilized product were compared).
The data obtained with respect to sodium alginate confirm the mucoadhesive feature of the analyzed fractions.

### Example 3

### Comparative Barrier Effect Assay

(For the barrier effect assay, two chambers physically separated by a semipermeable membrane (0.4 μm pores) were used. Human fibroblast cells were seeded in the bottom chamber, whereas the inflammatory agent LPS (purified E. Coli lipopolysaccharide) was introduced into the top chamber; on the semipermeable membrane separating the two chambers a thin film of the control incense extract or of incense fractions was stratified as described in the present invention.

Induced inflammatory reaction was estimated through semiquantitative dosage of Interleukin-6 (IL6) cytokine released in the culture medium of the bottom chamber: barrier effect assessment was obtained by comparison with the positive control in which the two chambers were separated by the same type of semipermeable membrane, free however from any barrier.)

As to the threshold value above which it may be said that a substance causes a barrier effect in the assay reported herein, a value equal to the 15% of inhibition with respect to the control was identified by the Inventors, during the setting up of the assay, on the basis of assays performed on substances known to have a barrier effect.

The experiment yielded the results reported in Table 1, which demonstrate that the incense extract fractions obtained create a barrier to LPS transit better than that created by an hydroalcoholic extract of 60% incense used as reference sample.

1. An incense extract fraction depleted in total boswellic acids, wherein said boswellic acids have a concentration expressed as weight percentage ≦1.3% or ≦0.1%.

2. The extract fraction according to claim 1 for use in protection and/or in repair of skin or mucous membranes.

3. The extract fraction according to claim 1, wherein said mucous membrane is oral mucosa, gastric mucosa, intestinal mucosa, nasal mucosa, vaginal mucosa, uterine mucosa, or rectal mucosa.

4. The extract fraction according to claim 1, wherein said incense extract is a hydroalcoholic extract.

5. A process for the preparation of extract fractions depleted in boswellic acids comprising the following steps:
a. grains of incense, optionally fragmented, are subjected to three or more steps of extraction in ethanol at decreasing concentrations, comprising at least one extraction with 85% ethanol, one extraction with 13% ethanol and one extraction with 3% ethanol;
b. the alcoholic extracts obtained with 13% ethanol and 3% ethanol in step a. are gathered and subjected to decantation and/or centrifugation;
c. the supernatant obtained after step b) is concentrated, obtaining a concentrated aqueous extract fraction, defined herein as fraction C, wherein said fraction C comprises boswellic acids at a concentration expressed as weight percentage ≦1.3%;
d. optionally, said fraction C is filtered; and
e. the permeate obtained in step d. is collected as fraction D and comprises boswellic acids at a concentration in weight percentage ≦0.1%.

6. The process according to claim 5, wherein said filtration at step d. comprises plural steps and is carried out on filters with a cutoff progressively reduced from 30 micrometers to about 0.1 micrometers.

7. A composition comprising one or more extract fractions according to claim 1.

8. The composition according to claim 7 for use in protection and/or in repair of skin or of mucous membranes.

9. The composition according to claim 8, wherein said mucous membrane is oral mucosa, gastric mucosa, intestinal mucosa, nasal mucosa, vaginal mucosa, uterine mucosa, or rectal mucosa.

10. The composition according to claim 7, further comprising substances of natural and/or plant origin having emollient, digestive, prokinetics, cholagogue, carminative, prebiotic, relaxing, excipient, preservative, and/or humectant properties.

11. The composition according to claim 10, wherein said substances of natural and/or plant origin are one or more selected from the group consisting of: extract of gentian root, boldo leaves extract, milk thistle fruits extract, artichoke leaves extract, dandelion root extract, anise fruit extract, rosemary leaf extract, mint leaves extract, marjoram leaves extract, cumin fruit extract, coriander fruit extract, ginger root extract, fennel fruit extract, caraway fruit extract, charcoal, and inulin.

12. The composition according to claim 7 in the form of capsule, tablet, lozenge, granule, powder, syrup, elixir, hard gelatine, soft gelatine, suspension, emulsion, solution, suppository, cream, gel, spray, ointment, salve, paste, oil-in-water emulsion, water-in-oil emulsion, oil-in-gel emulsion, or gel-in-oil emulsion.

13. The composition according to claim 7, wherein said composition is a pharmaceutical composition, is comprised in or consists of a medical device, is comprised in or consists of a medical food.

14. A medical device, drug, or medical food comprising a composition according to claim 7.

15. A method for the preparation of a composition according to claim 7, wherein an incense extract fraction depleted in total boswellic acids, wherein said boswellic acids have a concentration expressed as weight percentage ≦1.3% and/or an incense extract fraction depleted in total boswellic acids, wherein said boswellic acids have a concentration expressed as weight percentage ≦0.1%, is mixed with at least one of an excipient and/or a substance of natural and/or plant origin having emollient, digestive, prokinetics, cholagogue, carminative, prebiotic, relaxing, excipient, preservative, and/or humectant properties.

16. A pharmaceutical carrier comprising the extract fraction according to claim 1 characterized by promoting adhesion of active principles to mucous membranes.
